(19) United States
(12) Patent Application Publication
(10) Pub. No.: US 2023/0148921 A1
(43) Pub. Date: May 18, 2023

(54) Blood Collection Device

(71) Applicants: Thomas J. Shaw, Frisco, TX (US); Retractable Technologies, Inc., Little Elm, TX (US)

(72) Inventors: Thomas J. Shaw, Frisco, TX (US); Ni Zhu, McKinney, TX (US); Benjamin John Lardas, Denton, TX (US)

(21) Appl. No.: 17/526,918

(22) Filed: Nov. 15, 2021

(51) Int. Cl. A61B 5/15; A61B 5/153; A61B 5/151

(52) U.S. Cl. CPC A61B 5/15074; A61B 5/15003; A61B 5/150389; A61B 5/150732; A61B 5/153; A61B 5/150572; A61B 5/15192

(57) ABSTRACT

A blood collection device for medical use having a body with longitudinal flow channel and a needle retraction chamber, a slide assembly slidably engaging the body in a plane transverse to the flow channel through the body, a forwardly projecting nose, a device stabilization member with flexible wings projecting oppositely from a cylindrical hub attached to the nose, a rearwardly biased venipuncture needle projecting forwardly from the nose, and a tubular needle cap having proximal and distal ends, wherein the proximal end of the tubular needle cap is insertable between and is releasably held by the nose and the cylindrical hub. Cooperatively configured sets of ramps and stop surfaces limit lateral sliding movement of the body relative to the slide assembly.

### 1. FIELD OF THE INVENTION

This invention is a blood collection device for medical use having safety features that include a rearwardly biased venipuncture needle that is selectively retractable into a needle retraction chamber to reduce the likelihood of accidental needle sticks following use. A distinguishing feature of one embodiment of the invention is a tubular needle cap open at both ends that is releasably engageable with the inner wall of a hub portion of the flexible wing. Another distinguishing feature of one or more embodiments of the invention is an injection molded boss projecting outwardly from the nose of the slide member that is cooperatively configured and engageable with a window disposed in the hub of the flexible wing to seat the wing to the nose of the slide member during assembly of the device prior to packaging and use. Another distinguishing feature of one or more embodiments of the invention is an improvement to the cooperatively configured structures of the slide assembly and the body that facilitate sliding lateral movement of the body relative to the slide assembly during repositioning prior to needle retraction but prevent the slide member and the body from becoming disengaged accidentally prior to or following use

### 2. DESCRIPTION OF RELATED ART

Blood collection sets have previously been made with a closed-end needle cap that releasable engages a ridge, rim or seating surface on the nose of the barrel, and desirably remains in place until use of the device. Some devices have been made with needle caps that also comprise a projecting locking member insertable into a recess or needle retraction cavity to limit relative sliding movement between a frontal attachment and a body, and thereby prevent premature needle retraction that can render a device inoperable for the intended purpose prior to use. A prior art device with an integral tube seal disposed between the body and a laterally moveable slide member is disclosed, for example, in U.S. patent Ser. No. 10/646,148.

Notwithstanding the benefits and advantages achievable through the use of previously disclosed devices, a blood collection set is needed that desirably embodies: a flexible wing assembly providing a stable platform for drawing blood without allowing excessive needle movement causing discomfort to a patient (particularly children) during a blood draw; a needle cap preferably held in place by engagement with an inwardly facing, preferably elastomeric, surface of a hub portion of the flexible wing; and a slide member supporting the needle, needle cap, flexible wing and a rearwardly biased needle in lateral sliding engagement with a body comprising a rearwardly extending fluid path and a parallel needle retraction chamber into which the needle is retracted following use by sliding the body laterally relative to the slide member to release the rearwardly biased needle.

### SUMMARY OF THE INVENTION

A blood collection device is disclosed that desirably embodies: a flexible wing assembly providing a stable platform for drawing blood without allowing excessive needle movement causing discomfort to a patient (particularly children) during a blood draw; a needle cap preferably held in place by engagement with an inwardly facing, preferably elastomeric, surface of a hub portion of the flexible wing; and a slide assembly supporting the needle, needle cap, flexible wing and a rearwardly biased needle in lateral sliding engagement with a body comprising a rearwardly extending fluid flow path and a parallel, spaced-apart needle retraction chamber into which the needle is retracted following use by sliding the body laterally relative to the slide member to release the rearwardly biased needle.

This invention is a blood collection device for medical use having a rearwardly biased venipuncture needle that is selectively retractable into a needle retraction chamber to reduce the likelihood of accidental needle sticks following use. A distinguishing feature of the invention is a tubular needle cap open at both ends that is releasably engageable with the inner wall of a hub portion of the flexible wing. Another distinguishing feature of the invention is an injection molded boss projecting outwardly from the nose of the slide member that is cooperatively configured and engageable with a window disposed in the hub of the flexible wing to position and attach the wing to the nose of the slide member during assembly of the device prior to packaging and use. Another distinguishing feature of the invention is an improvement to the cooperatively configured ramps and stop surfaces of the slide member and the body that facilitate sliding lateral movement of the body relative to slide member during repositioning prior to needle retraction following a blood draw but prevent the slide member and the body from becoming disengaged prematurely and from reversing the direction of travel during repositioning.

Although the subject device is primarily envisioned for use in performing vascular blood draws, it will be apparent to those skilled in the art upon reading the disclosure in relation to the accompanying drawings that the subject device is also adaptable for use in drawing other bodily fluids.

### BRIEF DESCRIPTION OF THE DRAWINGS

The syringe of the invention is further described and explained in relation to the following drawings wherein:

FIG. 1 is an exploded view of a satisfactory embodiment of the blood collection set of the invention;

FIG. 2 is a top front perspective view of the assembled device of FIG. 1;

FIG. 3 is a top front perspective view as in FIG. 2 but with the needle cap removed;

FIG. 4 is top plan view of the device as in FIG. 2 with a segment of the blood collection tubing cut away for illustrative purposes;

FIG. 5 is left side elevation view of the device as in FIG. 4;

FIG. 6 is a rear elevation view of the device of FIG. 4;

FIG. 7 is a front elevation view of the device of FIG. 4;

FIG. 8 is a cross-sectional view taken along line 8-8 of FIG. 7;

FIG. 9 is a cross-sectional elevation view as in FIG. 8 but with the needle cap removed;

FIG. 10 is a cross-sectional view taken along line 10-10 of FIG. 4;

FIG. 11 is a view as in FIG. 4 with the needle cap removed and with the body repositioned laterally relative to the slide member and the needle retracted;

FIG. 12 is a view as in FIG. 5 but with the body repositioned laterally relative to the slide member and the needle retracted;

FIG. 13 is a view as in FIG. 7 but with the needle cap removed and the needle retracted;

FIG. 14 is a view as in FIG. 9 but with the body repositioned laterally relative to the slide member and the needle retracted;

FIG. 15 is a cross-section view as in FIG. 10 but with the body repositioned relative to the slide member and the needle retracted; and

FIG. 16 is an enlarged bottom perspective view, partially in section and partially cutaway, of the front portion of the device with the needle cap in place and prior to repositioning the body relative to the slide member to initiate needle retraction.

### DESCRIPTION OF PREFERRED EMBODIMENTS

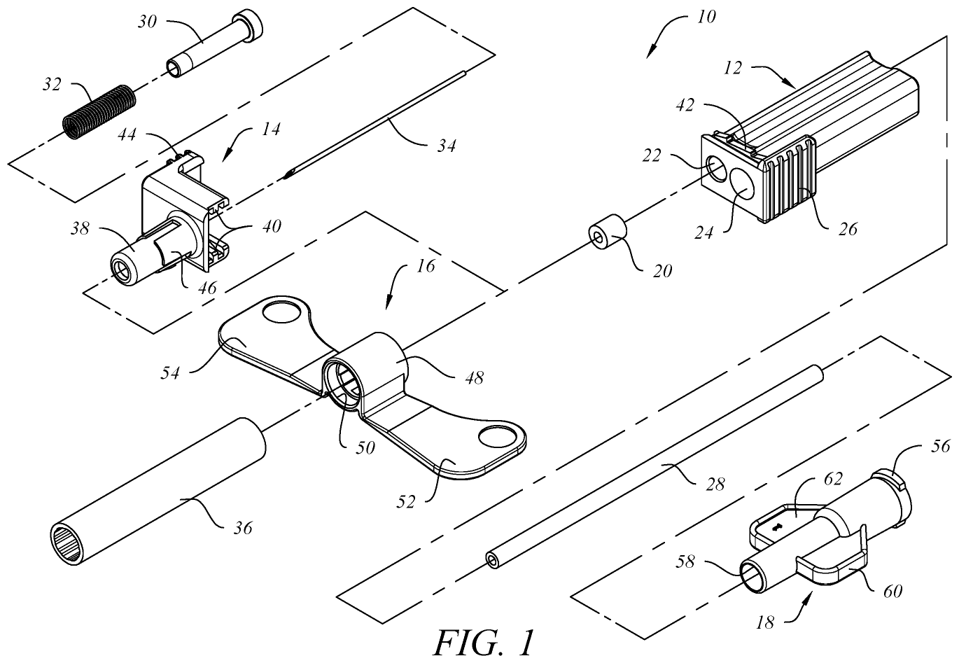

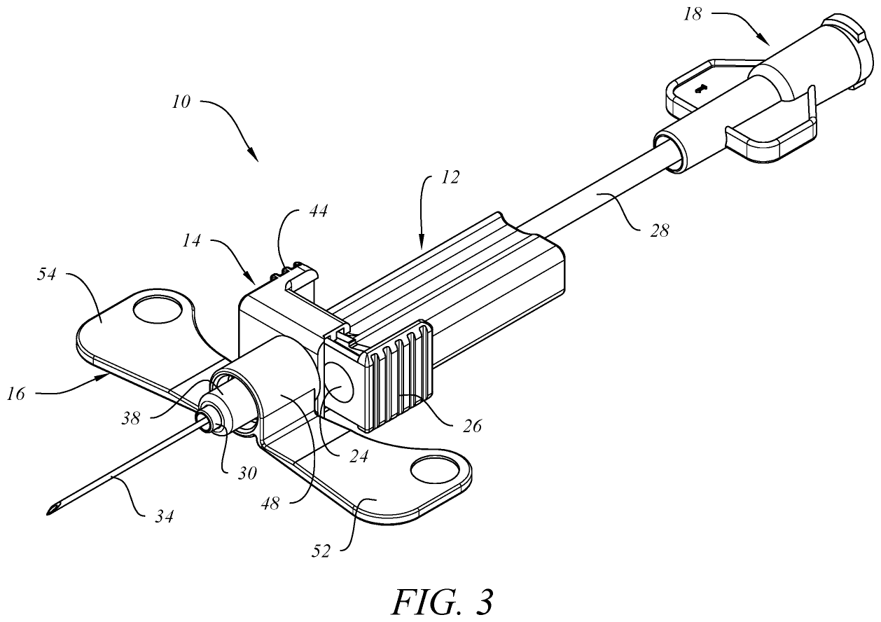

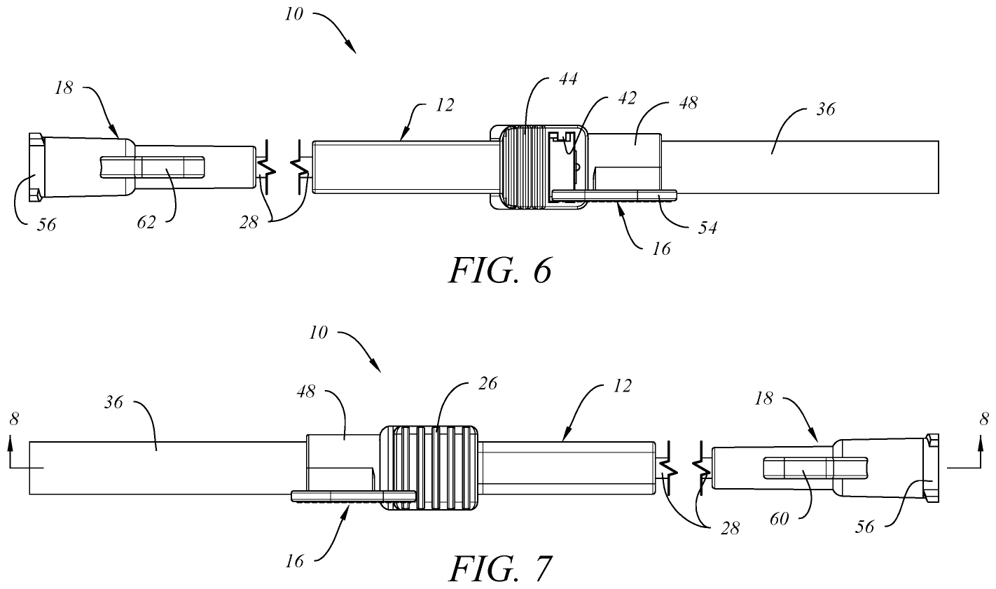

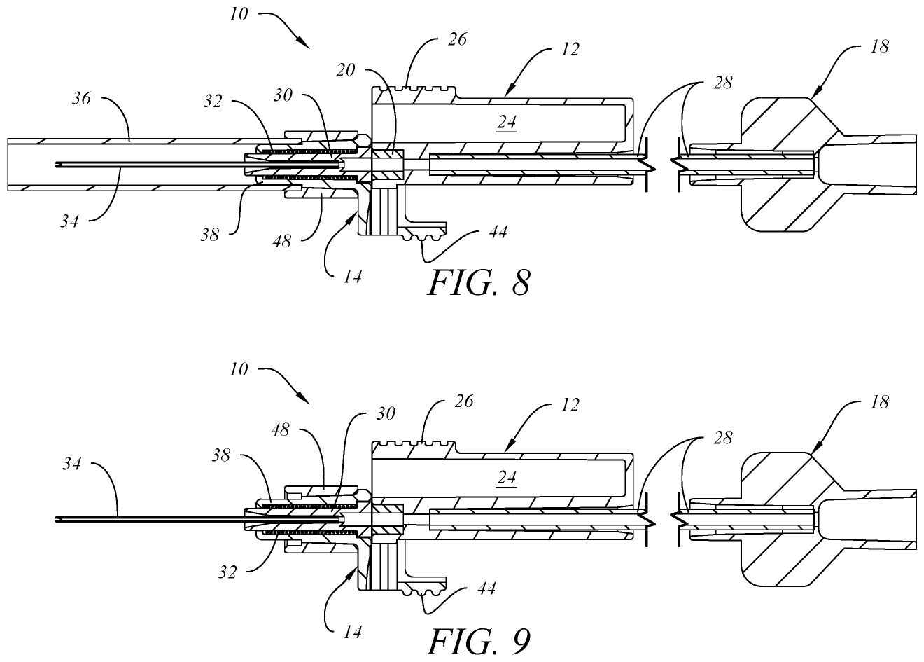

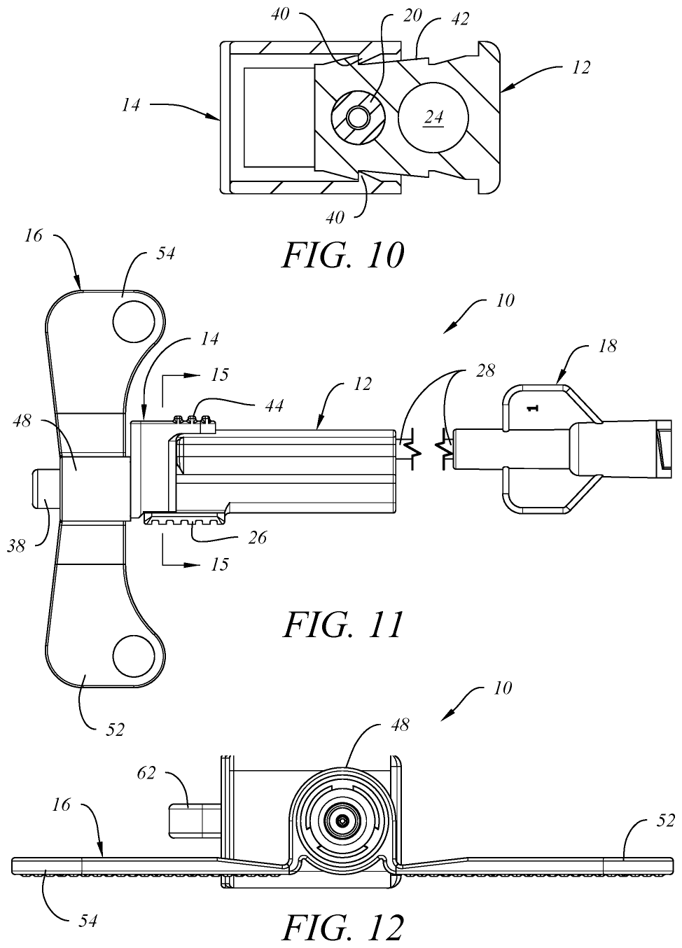

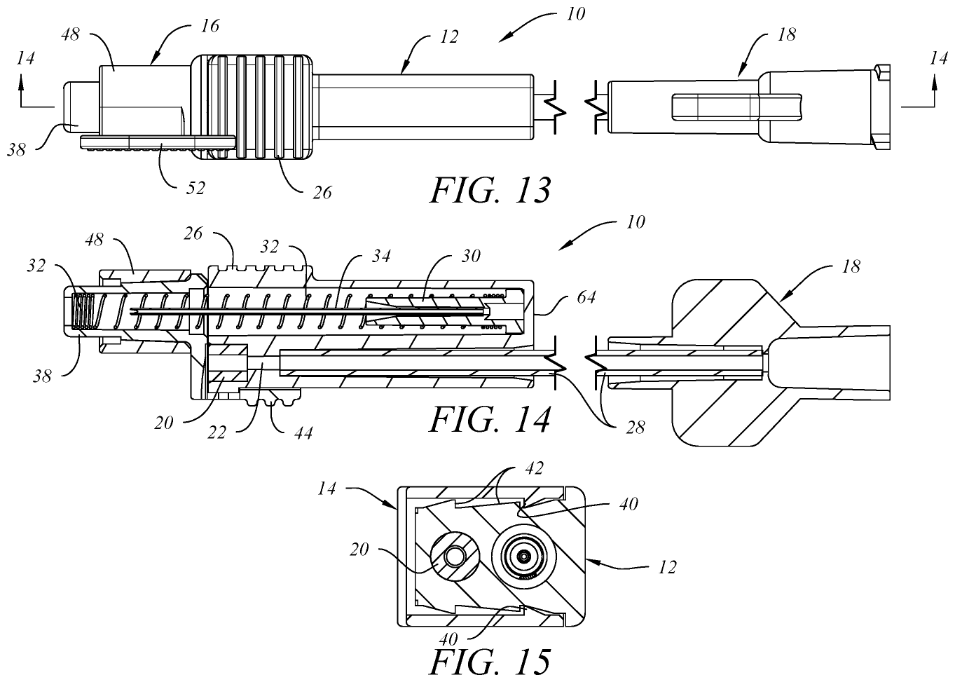

Referring to FIGS. 1-7, blood collection device 10 comprises body 12, slide assembly 14, device stabilization member 16, fluid flow tubing 28 with end connector fitting 18, and tubular needle cap 36. Body 12 satisfactorily comprises a longitudinally extending fluid flow channel 22 having proximal and distal ends and a parallel, laterally spaced-apart needle retraction chamber 24 having a closed proximal end and a distal open end. An annular fluid seal 20 with a longitudinal flow path coaxially aligned with fluid flow channel 22 is desirably made of a compressible elastomeric material, is satisfactorily seated inside a recess disposed in the distal open end of fluid flow channel 22 (visible in FIGS. 8-9). Body 12 further comprises upwardly and downwardly projecting structures 42 that are cooperatively configured with and slidably engage structures 40 of slide assembly 14 (visible in FIGS. 10-15). Textured touch surface 26 is desirably provided to facilitate selective lateral repositioning of body 12 relative to slide assembly 14 in a plane transverse to fluid flow channel 22 during use of blood collection device 10 as described in greater detail below. Body 12 of blood collection device 10 is preferably injection molded from a medical grade polymeric material that is transparent or sufficiently translucent to permit blood flowing through the body to be viewed by a user.

Slide assembly 14 desirably comprises an integrally molded plastic body further comprising projecting structures 40 configured to slidably engage structures 42 of body 12 to facilitate sliding lateral engagement between slide assembly 14 and body 12 as further described below. Slide assembly 14 further comprises a forwardly projecting nose 38, slotted collar 46 and touch surface 44. Touch surface 44 is preferably textured in the same manner as oppositely facing touch surface 26 of body 12 and facilitates selective repositioning of body 12 relative to slide assembly 14 during needle retraction following a blood draw, which is further discussed below. Referring particularly to FIG. 1, slide assembly 14 further comprises a compressed coil spring 32 that is seated inside the forwardly projecting nose 38 and has an inside diameter sufficient to allow the insertion of the distal shaft portion of needle holder 30 prior to attachment of slide assembly 14 to body 12 during assembly of blood collection device 10. Although forwardly projecting needle (or cannula) 34 is shown behind nose 38 in the “exploded” view of FIG. 1, the assembly of device 10 is preferably effectuated by inserting the blunt, proximal end of needle 34 into nose 38 and needle holder 30 after slide assembly 14 is attached to body 12 to hold spring 32 in compression during installation of needle 34. Attachment of needle 34 to needle holder 30 facilitates the formation of a continuous, coaxially aligned fluid flow path from needle 34 and needle holder 30, through annular fluid seal 20 and through fluid flow channel 22 into fluid flow tubing 28.

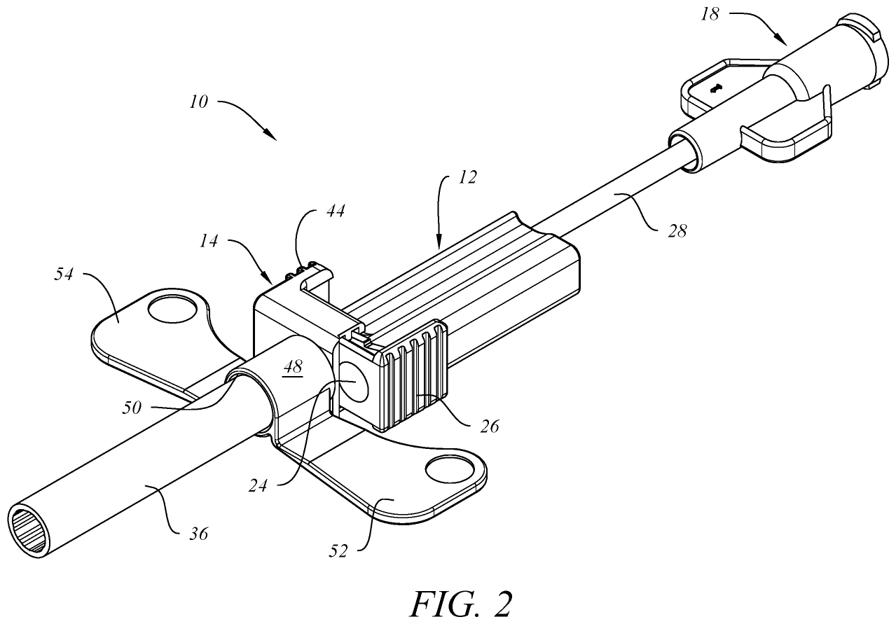

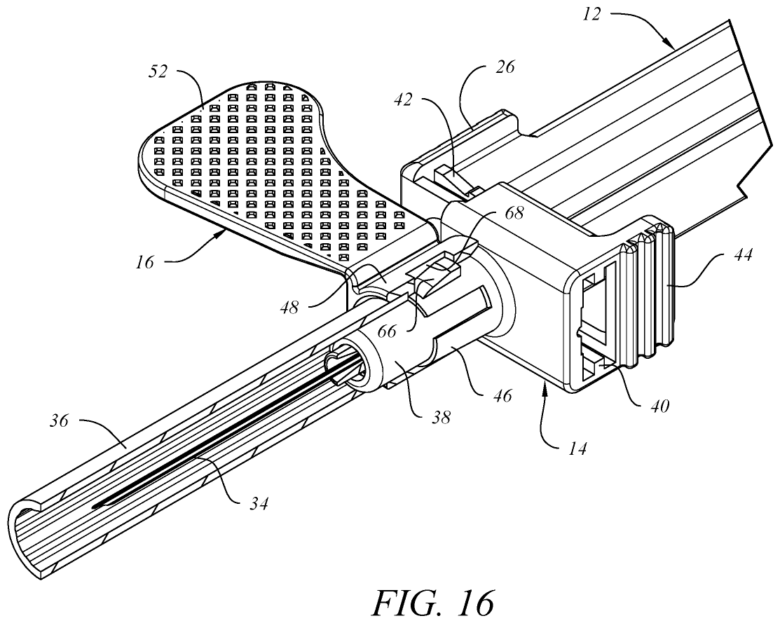

Device stabilization member 16 of blood collection device 10 is believed to be structurally different from and performs functions not common to “wings” of the type previously disclosed and used in other commercially available or prior art medical devices. Referring particularly to FIGS. 1 and 16, and more generally to FIGS. 2-7, device stabilization member 16 is desirably fabricated from a flexible polymeric material and preferably from an elastomeric material suitable for medical use for the intended purpose. Device stabilization member 16 is desirably integrally formed, although it can also be made as a composite construction including a molded plastic portion and an elastomeric or rubbery portion that are molded, coextruded, laminated, glued, welded or otherwise connected to form the desired overall structure. Principal elements of device stabilization member 16 include cylindrical hub 48 having an inside wall 50, and two oppositely projecting flexible wings 52, 54 that are adapted to rest on or be taped to a bodily surface following vascular insertion of needle 34 that projects forwardly through nose 38. When so used and secured, flexible wings 52, 54 help prevent the tip end of needle 34 from moving inside a vein or otherwise beneath the skin of a patient with whom blood collection device 10 is used. The downwardly facing surface of wings 52, 54 are desirably approximately level with the downwardly facing portion of cylindrical hub 48 and can be textured as shown in FIG. 16 if desired to help reduce the likelihood of slippage between the surfaces and underlying skin when blood collection device 10 is in use.

According to one preferred embodiment of slide assembly 14 and device stabilization member 16, an integrally molded boss 66 (visible in FIG. 16) projects outwardly from nose 38 through slotted collar 46, and is received through a pre-formed window 68 (also visible in FIG. 16) in cylindrical hub 48 to attach cylindrical hub 48 to slide assembly 14 and further stabilize slide assembly 14 and body 12 relative to forwardly projecting needle 34 and to the patient with whom device 10 is being used. Boss 66 prevents cylindrical hub 48 from rotating relative to slide member 14 and body 12, thereby cooperating with flexible wings projecting laterally from cylindrical hub 48.

Referring again to FIG. 1 end connector fitting 18 is disclosed as an example of one of the many commercially available fittings of the type that can be used with medical products to attach flexible fluid flow tubing 28 to a polymeric bag or other receptacle intended to receive the blood or other bodily fluid being drawn from a patient through device 10. The distal end of fluid flow tubing 28 can be inserted into frictional engagement with or otherwise attached to fluid flow channel 22 of body 12, and the proximal end of fluid flow tubing 28 can be inserted into frictional engagement with, or be otherwise connected to, distally facing cylindrical opening 58 of end connector fitting 18 that is in fluid communication with proximally facing luer connector 56 to complete a continuous fluid flow path through blood collection device 10. Two opposed, laterally extending wings 60, 62, are desirably provided to assist in maintaining a desired rotational alignment of end connector fitting 18 relative to body 12 and to device stabilization member 16. The continuous, substantially linear, fluid flow path passes through needle 34, needle holder 30 of slide assembly 14, annular fluid seal 20, fluid flow channel 22 of body 12, flexible fluid flow tubing 28 and end connector fitting 18 desirably remains open from the time of vascular insertion of needle 34 until body 12 is repositioned laterally relative to slide assembly 14 to initiate needle retraction as described in greater detail below.

Referring to FIGS. 1-3, 8 and 9, tubular needle cap 36 is desirably cylindrical, having a substantially constant diameter, and unlike many conventional needle caps for medical devices, is open at both the proximal and distal ends. The principal purposes of tubular needle cap 36 are to protect against accidental needle sticks and to protect the tip end of forwardly projecting needle 34 from being bent or blunted prior to use of the device. Because blood collective devices 10 are desirably individually packaged during manufacture, it is not necessary that the distal end be closed to prevent atmospheric contamination prior to use. The length of tubular needle cap 36 is satisfactorily such that the distal end extends sufficiently past the distal tip end of needle 34 that the needle tip is not reachable to produce an accidental needle stick by medical personnel unpackaging and handling blood collection device 10 prior to use. The polymeric material and wall thickness of tubular needle cap 36 are desirably selected and determined so that the needle cap is inflexible enough that it will not be accidentally flexed or bent into contact with needle 34 during normal handling and storage before it is removed just prior to use of device 10.

Unlike conventional needle caps used with similar medical devices, tubular needle cap 36 is not molded with one or more structural elements disposed at or near the proximal end that are specially designed and molded to frictionally engage and hold some part of the nose or body of a medical device. Instead, tubular needle cap 36 can be molded or extruded with a smooth, cylindrical inside and outside wall, provided that the inside and outside diameters permit the proximal end of tubular needle cap 36 to be installed over and around forwardly projecting nose 38 and inside the outwardly stretched, distal end of cylindrical hub 48 to be held in that position by a constrictive holding force exerted radially inward against the outer surface of tubular needle cap 36 by the elastomeric inside surface of cylindrical hub 48 of device stabilization member 16. During assembly of blood collection device 10, cylindrical hub 48 is desirably stretched or expanded outwardly for two purposes—first, to allow window 68 through cylindrical hub 48 (FIG. 16) to receive and be installed over and around projecting boss 66 of forwardly projecting nose 38 of slide assembly 14; and second, to allow the proximal end of tubular needle cap 36 to be inserted between nose 38 and inside the distal end of cylindrical hub 48. After cylindrical hub 48 and the proximal end of tubular needle cap 36 are properly positioned relative to forwardly projecting nose 38, cylindrical hub 48 is desirably allowed to relax and constrict to exert a holding force against the outside surface of the proximal end of tubular needle cap 36 that is sufficient to maintain the desired positional relationship between them until such time a tubular needle cap 36 is manually removed from the front of slide assembly 14 prior to use of blood collection device 10.

FIG. 8 illustrates blood collection device 10 in a fully assembled pre-use position wherein slide assembly 14 is disposed in lateral sliding engagement with body 12, the proximal end of needle 34 is installed inside needle holder 30, compressed spring 32 seated inside forwardly projecting nose 38 is biasing needle holder 30 rearwardly into abutting contact with the forwardly facing distal surface of annular fluid seal 20, the distal end of flexible fluid flow tubing 28 is installed inside fluid flow channel 22 of body 12, and the proximal end of fluid flow tubing 28 is installed inside end connector fitting 18. As best seen in FIG. 8, whenever tubular needle cap 36 is installed over needle 34 and is inserted into engagement between forwardly projecting tubular nose 38 and the forwardly extending portion of cylindrical hub 48 of device stabilization member 16, the elastomeric holding force exerted against the outwardly facing surface of tubular needle cap 36 is desirably such that tubular needle cap 36 is held in place relative to slide assembly 14 and body 12 until such time as tubular needle cap 36 is intentionally removed prior to use of blood collection device 10. When blood collection device 10 is configured as shown in FIG. 8, a continuous, substantially linear, coaxially aligned fluid flow path is established from needle 34 to and through end connector fitting 18 that is accessible by removing tubular needle cap 36 as shown in FIG. 9. Once tubular needle cap 36 is removed from the front of blood collection device 10 prior to vascular insertion of needle 34, tubular needle cap 36, which is not contaminated, can be disposed of in the trash without special safeguards.

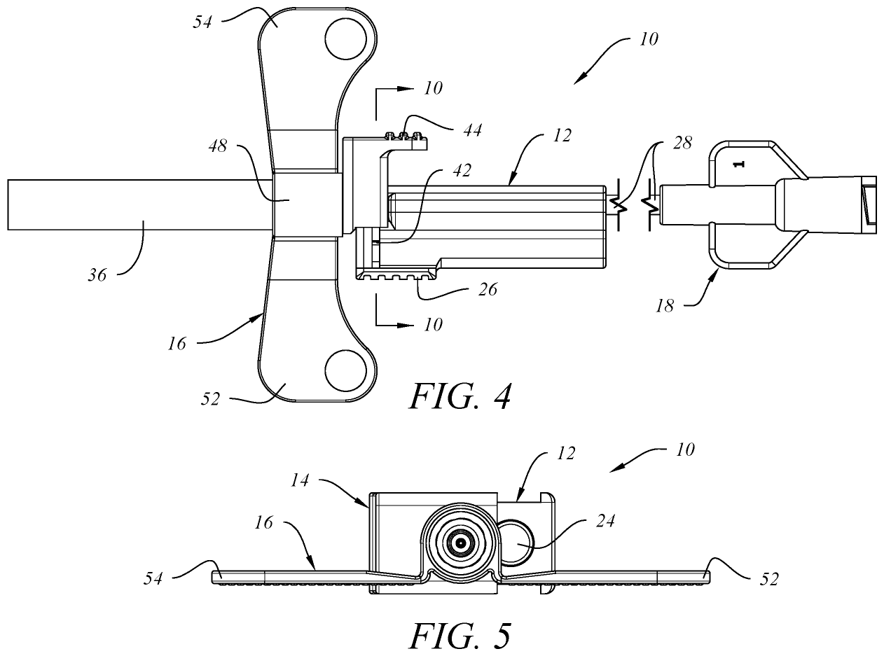

FIGI. 10 is a cross-section taken along line 10-10 of FIG. 4 and shows the lateral sliding engagement of body 12 with slide assembly 14 when body 12 is disposed in the pre-use position where tubular needle cap 36 is still attached and aligned with annular fluid seal 20 in a forwardly facing recess of fluid flow channel 22. The leading, first set of inclined top and bottom ramps 42 of body 12 is desirably configured to slide past top and bottom ramps 40 of slide assembly 14 during assembly of blood collection device 10, and the oppositely facing shoulders of ramps 40 desirably prevent body 12 from moving in a reverse direction relative to slide assembly 14 and accidentally disengaging prior to use of blood collection device 10. FIGS. 4-10 and 16 all disclose blood collection device 10 with a coaxially aligned fluid flow path between needle 34, annular fluid seal 20, fluid flow tube 28 and end connector fitting 18. With blood collection device 10 disposed in this position, and following the selective removal of tubular needle cap 36, needle 34 can be inserted into a patient and if desired, wings 52, 54 can be taped to hold device 10 steady as blood is collected through fluid flow tube 28. Because body 12 and flu id flow tub 28 are preferably either transparent or translucent, a user can observe blood flowing through body 12 to confirm proper vascular insertion of needle 34 in the patient.

Following the blood draw, body 12 is desirably repositioned transversely relative to the fluid flow direction and laterally relative to slide assembly 14 as shown in FIGS. 11-15 by exerting manual pressure against touch surfaces 26, 44 of slide assembly 14, thereby causing the second set of top and bottom ramps 42 of body 12 to slide laterally past top and bottom ramp 40 of slide assembly 14, after which they are prevented by the facing and contacting shoulders from reversing direction subsequent to the repositioning. As touch surfaces 26, 44 are squeezed toward each other by the user, the proximal end of needle holder 30 is moved laterally out of abutting contact with the distal end of annular fluid seal 20 and fluid seal 20 expands into facing contact with the rearwardly facing surface of slide assembly 14. This contact helps prevent collected bodily fluid from leaking forwardly out of fluid flow path 22. At the same time, the larger diameter opening into needle retraction chamber 24 becomes aligned with the smaller diameter head of needle holder 30, allowing compressed spring 32 to drive needle holder 30 and needle 34 rearwardly into needle retraction chamber 24. Tubular nose 38 of slide assembly 14, needle 34, needle holder 30 and relaxed compression spring 32 and needle retraction chamber 24 are desirably sized and cooperatively configured so that the distal tip end of needle 34 does not extend forwardly of tubular nose 28 after needle holder 30 is driven rearwardly into needle retraction chamber 24. Blood collection device 10 is thereby prevented from causing accidental needle sticks by protecting the distal end of needle 34 inside forwardly projecting nose 38 following needle retraction, and further use of the device is also prevented due to bridging of the lateral sliding surfaces by expanded spring 32 and by the irreversibly engaged shoulder surfaces of ramps 40, 42 between the facing portions of slidably engaged slide assembly 14 and body 12.

It will become apparent to those of ordinary skill in the art upon reading this specification in relation to the accompanying drawings that various other modifications and alterations can also be made to the embodiments disclosed here, and it is intended that the scope of the invention be limited only by the broadest interpretation of the appended claims to which the inventors are legally entitled.

We claim:

1. A blood collection device for medical use, the device comprising: a body; a slide assembly disposed in laterally slidable engagement with the body and having a forwardly projecting nose; a device stabilization member with flexible wings projecting oppositely from a cylindrical hub attached to the nose; a rearwardly biased venipuncture needle projecting forwardly from the nose; and a tubular needle cap having proximal and distal ends; wherein the proximal end of the tubular needle cap is insertable between and is releasably held by the nose and the cylindrical hub.

2. The blood collection device of claim 1 wherein the tubular needle cap is supported by the nose and is releasably held by frictional engagement with an inwardly facing surface of the cylindrical hub.

3. The blood collection device of claim 2 wherein the cylindrical hub comprises an elastomeric material that exerts a holding force around the proximal end of the tubular needle cap.

4. The blood collection device of claim 1 wherein the nose further comprises an outwardly projecting boss and the cylindrical hub comprises a window configured to receive the boss to position and attach the cylindrical hub to the slide assembly.

5. The blood collection device of claim 2 wherein the tubular needle cap is releasably held by frictional engagement with both the nose and the cylindrical hub.

6. The blood collection device of claim 1 wherein the body comprises a longitudinally extending cylindrical flow channel having proximal and distal ends, and a needle retraction chamber that is parallel to and laterally spaced apart from the cylindrical flow channel.

7. The blood collection device of claim 6 wherein the needle retraction chamber has an open distal end and a closed proximal end.

8. The blood collection device of claim 6, further comprising a compressible annular fluid seal seated inside the distal end of the cylindrical flow channel in abutting engagement with a rearwardly facing surface portion of the slide assembly.

9. The blood collection device of claim 1 wherein the slide assembly comprises a needle retraction assembly.

10. The blood collection device of claim 9 wherein the needle retraction assembly comprises a tubular needle holder and a compressed needle retraction spring surrounding a forwardly extending portion of the tubular needle holder, the compressed needle retraction spring having a distal end seated inside the nose and a proximal end abutting against a forwardly facing annular shoulder of the needle holder.

11. The blood collection device of claim 10 wherein the tubular needle holder has a proximal end that is coaxially aligned with and abuts against an annular fluid seal disposed inside a fluid flow channel in the body.

12. The blood collection device of claim 6 further comprising a section of flexible tubing projecting rearwardly from a proximal end of the cylindrical flow channel, the flexible tubing providing fluid communication with an end fitting connectable to a receptacle for collected blood.

13. The blood collection device of claim 1 wherein the slide assembly comprises a first set of upper and lower ramps and the body comprises a second set of oppositely configured upper and lower ramps, wherein the first set and second sets of upper and lower ramps are each slidably engageable to allow relative sliding movement of the body toward the slide assembly to initiate needle retraction.

14. The blood collection device of claim 13 wherein the slide assembly comprises a first set of upper and lower stop surfaces adjacent to the first set of upper and lower ramps and the body comprises a second set of oppositely facing upper and lower stop surfaces adjacent to the second set of second set of oppositely configured upper and lower ramps, wherein the first set and second sets of upper and lower stop surfaces are positioned to abut each other to prevent disengagement of the body from the slide assembly prior to needle retraction and to prevent relative sliding movement of the body away from the slide assembly following needle retraction.

15. The blood collection device of claim 1 wherein the body of the device is molded from a medical grade polymeric material that is transparent or sufficiently translucent to permit blood flowing through the body to be viewed by a user.
